(19) United States
(12) Patent Application Publication
(10) Pub. No.: US 2024/0307300 A1
(43) Pub. Date: Sep. 19, 2024

(54) AN IMPROVED PROCESS FOR THE PREPARATION OF AN AQUEOUS OPHTHALMIC SOLUTION OF DIFLUPREDNATE

(71) Applicant: SUN PHARMA ADVANCED RESEARCH COMPANY LIMITED, Mumbai (IN)

(72) Inventors: Arindam HALDER, Vadodara (IN); Ajay Jaysingh KHOPADE, Vadodara (IN)

(21) Appl. No.: 18/681,812

(22) PCT Filed: Oct. 7, 2021
(86) PCT No.: PCT/IB2021/059213

§ 371 Date: Feb. 6, 2024

(30) Foreign Application Priority Data
Aug. 25, 2021 (IN) 202121038494

(51) Int. Cl. A61K 9/00; A61K 31/573; A61K 47/02; A61K 47/10; A61K 47/12; A61K 47/18; A61K 47/32; A61K 47/34; A61K 47/44

(52) U.S. Cl. CPC A61K 9/0048; A61K 31/573; A61K 47/02; A61K 47/10; A61K 47/12; A61K 47/183; A61K 47/186; A61K 47/32; A61K 47/34; A61K 47/44

(57) ABSTRACT

The present invention relates an improved process for the preparation of an aqueous ophthalmic solution of difluprednate or pharmaceutically acceptable salts thereof. The present invention further relates to an aqueous ophthalmic solution of difluprednate or pharmaceutically acceptable salts thereof, prepared by the improved process of the present invention.

### FIELD OF THE INVENTION

The present invention relates an improved process for the preparation of an aqueous ophthalmic solution of difluprednate or pharmaceutically acceptable salts thereof. The present invention further relates to an aqueous ophthalmic solution of difluprednate or pharmaceutically acceptable salts thereof, prepared by the improved process of the present invention.

### BACKGROUND OF THE INVENTION

Difluprednate is an anti-inflammatory corticosteroid drug represented by formula I below:

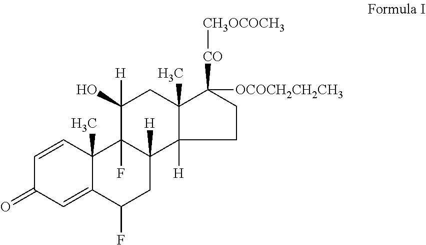

Difluprednate, a steroidal drug is practically insoluble in aqueous vehicle. The currently approved formulation of difluprednate in the United States is marketed under the brand name of DUREZOL® which is an emulsion dosage form and not an aqueous solution. Durezol® comprises 0.05% w/v difluprednate emulsified between castor oil and water. Durezol® emulsion formulation is indicated for the treatment of inflammation and pain associated with ocular surgery and endogenous anterior uveitis when administered four times a day. As the emulsion needs to be instilled four times-a-day, there are high chances of patient non-compliance and missing a dose.

U.S. Pat. No. 6,114,319 (herein after referred to as the '319 patent) describes an oil-in-water emulsion formulation of difluprednate, which contains a fatty acid ester of glycerol as an oil and non-ionic surfactant as emulsifier. The process according to the '319 patent comprises: preparation of an oil phase comprising difluprednate and an aqueous phase comprising the surfactant followed by addition of an oil phase to the aqueous phase.

U.S. Pat. No. 5,556,848 (herein after referred to as the '848 patent) describes an aqueous suspension formulation of difluprednate and a non-ionic surfactant as dispersion stabilizer. The process according to the '848 patent comprises suspending difluprednate in an aqueous solution comprising the non-ionic surfactant.

U.S. Pat. No. 11,000,475 (herein after referred to as the '475 patent) describes an ophthalmic solution of difluprednate comprising a crystal growth inhibitor. The ophthalmic solution of difluprednate according to the '475 was prepared by the process as described in the examples 1-4 therein.

While scaling up the process for the preparation of the aqueous solution of difluprednate, the inventors of the present invention observed excessive foaming. Due to the foaming, the inventors faced critical issues in terms of unpredictable and inaccurate volume measurement. Further, when the solution was filtered or allowed to settle-down the foam, it resulted to high yield loss, increased batch processing time and thus increased operational cost.

### SUMMARY OF THE INVENTION

The inventors have surprisingly identified an improved process which avoids excessive foaming during the commercial manufacturing of the aqueous ophthalmic solution of difluprednate.

In the first aspect, the present invention relates to a process for the preparation of an aqueous ophthalmic solution of difluprednate having a pH of 4.5 to 5.5 and an osmolality from about 95 to about 150 mOsm/kg, comprising:
a. preparation of a non-aqueous drug phase by dissolving difluprednate and benzalkonium chloride in polyoxyl 35 castor oil:
b. preparation of an aqueous phase comprising:
   i. preparing a polymer phase by dissolving polyvinyl alcohol in water for injection:
   ii. preparing a buffer phase by dissolving glacial acetic acid, glycerin, boric acid, disodium edetate, sodium acetate and N-lauryl sarcosine sodium in water for injection at a stirring rate of about 200 to about 400 rotations per minute; and
   iii. addition of buffer phase to the polymer phase, wherein the addition is done at a stirring rate of about 200 to about 600 rotations per minute;
c. preparation of a bulk solution by addition of the non-aqueous drug phase to the aqueous phase; and
d. addition of polyhexamethylene biguanide to the bulk solution to achieve the aqueous ophthalmic solution of difluprednate.

In a second aspect, the present invention relates to an aqueous ophthalmic solution of difluprednate having a pH of 4.5 to 5.5 and an osmolality from about 95 to about 150 mOsm/kg, comprising:
i. about 0.03% w/v to about 0.04% w/v difluprednate;
ii. about 0.5% w/v to about 3.0% w/v polyvinyl alcohol or a derivative thereof;
iii. about 0.01% w/v to about 0.05% w/v benzalkonium chloride; and
iv. about 1.5% w/v to about 6.0% w/v polyoxyl 35 castor oil;
   wherein the said aqueous ophthalmic solution is prepared by the process comprising:
a. preparation of a non-aqueous drug phase by dissolving difluprednate and benzalkonium chloride in polyoxyl 35 castor oil;
b. preparation of an aqueous phase comprising:
   i. preparing a polymer phase by dissolving polyvinyl alcohol in water for injection;
   ii. preparing a buffer phase by dissolving glacial acetic acid, glycerin, boric acid, disodium edetate, sodium acetate and N-lauryl sarcosine sodium in water for injection at a stirring rate of about 200 to about 400 rotations per minute; and
   iii. addition of the buffer phase to the polymer phase, wherein the addition is done at a stirring rate of about 200 to about 600 rotations per minute;
c. preparation of a bulk solution by addition of non-aqueous drug phase to aqueous phase; and
d. addition of polyhexamethylene biguanide to the bulk solution to achieve the aqueous ophthalmic solution of difluprednate.

### DETAILED DESCRIPTION OF THE INVENTION

The “aqueous ophthalmic solution” as stated herein, is a solution of difluprednate in an aqueous vehicle, wherein difluprednate is in the solubilized form and not in particulate form, either microparticulate or nanoparticulate or in micellar form.

The word ‘difluprednate’ as used herein includes prodrugs of difluprednate wherein the hydroxyl group in difluprednate is converted to a labile ester or an amide.

In a first aspect, the present invention relates to a process for the preparation of an aqueous ophthalmic solution of difluprednate having a pH of 4.5 to 5.5 and an osmolality from about 95 to about 150 mOsm/kg, comprising:
a. preparation of a non-aqueous drug phase by dissolving difluprednate and benzalkonium chloride in polyoxyl 35 castor oil;
b. preparation of an aqueous phase comprising:
   i. preparing a polymer phase by dissolving polyvinyl alcohol in water for injection;
   ii. preparing a buffer phase by dissolving glacial acetic acid, glycerin, boric acid, disodium edetate, sodium acetate and N-lauryl sarcosine sodium in water for injection at a stirring rate of about 200 to about 400 rotations per minute; and
   iii. addition of the buffer phase to the polymer phase, wherein the addition is done at a stirring rate of about 200 to about 600 rotations per minute;
c. preparation of a bulk solution by addition of the non-aqueous drug phase to the aqueous phase; and
d. addition of polyhexamethylene biguanide to the bulk solution to achieve the aqueous ophthalmic solution of difluprednate.

In one embodiment of the present disclosure, the process further comprises addition of water for injection in order to make up the volume according to the batch size.

In another embodiment of the present disclosure, the process further comprises filtration through a sterile 0.2 micron filter. In a preferred embodiment, the solution is filtered through a hydrophilic modified polyvinylidene fluoride (PVDF) filter, available under the brand name Fluorodyne® II DFL capsule filters having pore size 0.2 micron.

Upon considerable efforts and evaluating various process parameters, the inventors believe that stirring rate is an important parameter to avoid foaming. It was further identified that by controlling the stirring rate during preparation of an aqueous phase, any foaming issue is prevented. Particularly during the preparation of the buffer phase, when N-lauryl sarcosine sodium is added, uncontrolled stirring causes excessive foaming, which is then carried forward and increases upon addition of the buffer phase to the polymer phase and further to the final bulk phase and finished aqueous solution of difluprednate. Thus, it is important to prevent foaming during the preparation of the aqueous phase, specifically during the preparation of the buffer phase and addition thereof to the polymer phase.

In one embodiment of the present disclosure, the buffer phase is prepared at a stirring rate not more than 400 rotations per minute. In a preferred embodiment, the buffer phase is prepared at a stirring rate of about 200 to about 400 rotations per minute.

In one preferred embodiment, the addition of N-lauryl sarcosine sodium in the buffer phase preparation is done at a stirring rate not more than 400 rotations per minute. In a preferred embodiment, said addition of N-lauryl sarcosine sodium is done at a stirring rate of about 200 to about 400 rotations per minute.

In another embodiment of the present disclosure, the addition of the buffer phase to the polymer phase is done at a stirring rate not more than 600 rotations per minute. In a preferred embodiment, said addition is done at a stirring rate of about 200 to about 600 rotations per minute.

In one preferred embodiment of the present disclosure, the aqueous ophthalmic solution of difluprednate comprises:
i. about 0.03% w/v to about 0.04% w/v difluprednate;
ii. about 0.5% w/v to about 3.0% w/v polyvinyl alcohol or derivative thereof;
iii. about 0.01% w/v to about 0.05% w/v benzalkonium chloride; and
iv. about 1.5% w/v to about 6.0% w/v polyoxyl 35 castor oil;

In another embodiment of the present disclosure, wherein the aqueous ophthalmic solution of difluprednate further comprises:
v. about 0.002% w/v to about 0.02% w/v polyhexamethylene biguanide;
vi. about 0.02% w/v to about 0.05% w/v N-lauroyl sarcosine sodium;
vii. about 0.05% w/v to about 1.5% w/v boric acid;
viii. about 0.01% w/v to about 0.1% w/v disodium edetate; and
ix. about 1.0% w/v to about 3.0% w/v glycerine.

In a second aspect, the present invention relates to an aqueous ophthalmic solution of difluprednate having a pH of 4.5 to 5.5 and an osmolality from about 95 to about 150 mOsm/kg, comprising:
i. about 0.03% w/v to about 0.04% w/v difluprednate;
ii. about 0.5% w/v to about 3.0% w/v polyvinyl alcohol or derivative thereof;
iii. about 0.01% w/v to about 0.05% w/v benzalkonium chloride; and
iv. about 1.5% w/v to about 6.0% w/v polyoxyl 35 castor oil;
   wherein the said aqueous ophthalmic solution is prepared by the process comprising:
a. preparation of a non-aqueous drug phase by dissolving difluprednate and benzalkonium chloride in polyoxyl 35 castor oil;
b. preparation of an aqueous phase comprising:
   i. preparing a polymer phase by dissolving polyvinyl alcohol in water for injection;
   ii. preparing a buffer phase by dissolving glacial acetic acid, glycerin, boric acid, disodium edetate, sodium acetate and N-lauryl sarcosine sodium in water for injection at a stirring rate of about 200 to about 400 rotations per minute; and
   iii. addition of the buffer phase to the polymer phase, wherein the addition is done at a stirring rate of about 200 to about 600 rotations per minute;
c. preparation of a bulk solution by addition of the non-aqueous drug phase to the aqueous phase; and
d. addition of polyhexamethylene biguanide to the bulk solution to achieve the aqueous ophthalmic solution of difluprednate.

In one embodiment of the present disclosure, the concentration (% weight by volume or % w/v) of difluprednate is expressed in terms of difluprednate base, and is present at a concentration that ranges from about 0.005% w/v to about 0.07% w/v, preferably from about 0.02% w/v to about 0.045% w/v, more preferably from about 0.03% w/v to about 0.04% w/v.

In another embodiment of the present disclosure, the aqueous ophthalmic solution comprises a mixture of a quaternary ammonium compound and polyethoxylated castor oil as a solubilizer.

In one embodiment of the present disclosure, the quaternary ammonium compound that is used in the mixture as a solubilizer is preferably benzalkonium chloride. According to another embodiment, the benzalkonium chloride is present in an amount ranging from about 0.0002% w/v to about 0.08% w/v, preferably from about 0.01% w/v to about 0.05% w/v.

In one embodiment, the polyethoxylated castor oil that is used in the mixture as a solubilizer is preferably polyoxyl 35 castor oil, marketed under the tradename Cremophor® EL by BASF Corp. According to another embodiment, the polyoxyl 35 castor oil is present in an amount ranging from about 1.9% w/v to about 10% w/v, preferably from about 1.5% w/v to about 6.0% w/v.

In one particularly preferred embodiment, the aqueous ophthalmic solution comprises a mixture of benzalkonium chloride and polyoxyl 35 castor oil as solubilizer.

In another embodiment of the present disclosure, the aqueous ophthalmic solution comprises polyvinyl alcohol or its derivatives as a crystal growth inhibitor. In one specific embodiment, polyvinyl alcohol is present in the range from about 0.1% w/v to about 5.0% w/v, preferably from about 0.5% w/v to about 3.0% w/v.

The aqueous solution of the present invention may further include, other conventional excipients such as preservatives, chelating agents, co-solvents, buffers and so on.

In one preferred embodiment, the aqueous ophthalmic solution comprises one or more preservatives selected from a polyhexamethylene biguanide, boric acid, N-lauroyl sarcosine sodium or mixtures thereof. In one particularly preferred embodiment, the aqueous ophthalmic solution of difluprednate comprises a mixture of polyhexamethylene biguanide, boric acid and N-lauroyl sarcosine sodium as preservative.

In one preferred embodiment, the aqueous ophthalmic solution comprises polyhexamethylene biguanide in an amount ranging from about 0.001% w/v to about 0.04% w/v, preferably from about 0.002% w/v to about 0.02% w/v, more preferably for example 0.005% w/v.

In another preferred embodiment, the aqueous ophthalmic solution comprises boric acid in an amount ranging from about 0.05% w/v to about 1.5% w/v, preferably for example, 0.6% w/v.

In another preferred embodiment, the aqueous ophthalmic solution comprises N-lauroyl sarcosine sodium in an amount ranging from about 0.001% w/v to about 0.5% w/v, preferably from about 0.02 w/v to about 0.05% w/v, more preferably for example 0.03% w/v.

In one preferred embodiment, the aqueous ophthalmic solution comprises disodium edetate as a chelating agent present in an amount ranging from about 0.001% w/v to about 0.5% w/v, preferably from about 0.01% w/v to about 0.1% w/v, more preferably for example 0.05% w/v.

In one preferred embodiment, the aqueous ophthalmic solution comprises glycerine as a co-solvent present in an amount ranging from about 0.5% w/v to about 5.0% w/v, preferably from about 1.0% w/v to about 3.0% w/v, preferably for example 1.6% w/v of the solution.

In one preferred embodiment, the aqueous ophthalmic solution comprises acetic acid, sodium acetate or mixtures thereof as a pH adjusting agent and/or buffer. In another preferred embodiment, the pH adjusting agent and/or buffer are added in quantities sufficient to achieve a pH of the aqueous ophthalmic solution from about 4.5 to about 5.5.

The said aqueous solution has an osmolality from about 95 to about 150 mOsm/kg, preferably from about 100 to about 130 mOsm/kg, more preferably from about 115 to about 125 mOsm/kg.

In one embodiment, the present invention relates to an aqueous ophthalmic solution of difluprednate having a pH of 4.5 to 5.5 and an osmolality from about 95 to about 150 mOsm/kg.

In one preferred embodiment of the present disclosure, the aqueous ophthalmic solution of difluprednate comprises:
i. about 0.03% w/v to about 0.04% w/v difluprednate;
ii. about 0.5% w/v to about 3.0% w/v polyvinyl alcohol or derivative thereof;
iii. about 0.01% w/v to about 0.05% w/v benzalkonium chloride; and
iv. about 1.5% w/v to about 6.0% w/v polyoxyl 35 castor oil;

In another embodiment of the present disclosure, wherein the aqueous ophthalmic solution of difluprednate further comprises:
v. about 0.002% w/v to about 0.02% w/v polyhexamethylene biguanide;
vi. about 0.02% w/v to about 0.05% w/v N-lauroyl sarcosine sodium;
vii. about 0.05% w/v to about 1.5% w/v boric acid;
viii. about 0.01% w/v to about 0.1% w/v disodium edetate; and
ix. about 1.0% w/v to about 3.0% w/v glycerine.

In another embodiment, the present invention relates to an aqueous ophthalmic solution of difluprednate of above embodiments, prepared by the process according to the first aspect.

While the present invention is disclosed generally as above, additional aspects are further discussed and illustrated with reference to the example below. However, the example is presented merely to illustrate the invention and should not be considered as a limitation thereto.

### EXAMPLE

The example describes an improved process for the preparation of an aqueous ophthalmic solution of difluprednate according to the present invention.

TABLE 1
Aqueous ophthalmic solution of difluprednate:

| Ingredients | Example 1 |
|---|---|
| Difluprednate | 20 g |
| Benzalkonium chloride | 12.5 g |
| N-lauroyl sarcosine sodium | 15 g |
| Polyoxyl 35 castor oil (Cremophor ® EL) | 2500 g |
| Polyvinyl alcohol | 700 g |
| Glycerine | 750 g |
| Boric acid | 300 g |
| Poly hexa methylene biguanide | 2.5 g |
| Disodium edetate | 25 g |
| Sodium acetate trihydrate | 5 g |
| Glacial acetic acid | 2.5 g |
| Water for Injection | q.s to 50 L |

### Stage A: Preparation of a Non-Aqueous Drug Phase

Polyoxyl 35 castor oil (500 mL) was added to difluprednate (20 g) followed by addition of benzalkonium chloride (12.5 g as 50% solution). The mixture was further added to additional polyoxyl 35 castor oil (500 mL) and stirred until the difluprednate was completely dissolved.

### Stage B: Preparation of an Aqueous Phase

i. Preparation of a polymer phase: Polyvinyl alcohol (700 g) was dissolved in water for injection (21 L) under stirring at 70-80°° C. The resulting solution was then cooled at 40-45° C. and eventually at 20-25° C.

ii. Preparation of a buffer phase: Sodium acetate trihydrate (5 g), Glacial acetic acid (2.5 g), glycerine (750 g), boric acid (300 g) and disodium edetate (25 g) were dissolved in water for injection (13 L). The resulting solution was slowly added to N-lauryl sarcosine sodium (15 g as 30% solution) and stirred under controlled stirring at about 200 to about 400 rotations per minute to obtain the buffer phase.

iii. The buffer phase was then added to the polymer phase, again under controlled stirring at about 200 to about 600 rotations per minute, followed by addition of water for injection (2 L) to obtain the aqueous phase.

### Stage C: Preparation of a Bulk Solution

The non-aqueous drug phase of Stage A was added to the aqueous phase of Stage B followed by the addition of water for injection (3 L). The resulting bulk solution was stirred until a uniform mixture was formed.

### Stage D: Preparation of Final Aqueous Solution

Polyhexamethylene biguanide (2.5 g as 20% solution) was added to the bulk solution of Stage C. Subsequently, volume make up was carried out using water for injection up to 50 L. The resulting solution was filtered through a sterile 0.2 μm PVDF capsule filter to obtain the final aqueous ophthalmic solution of difluprednate.

The resulting aqueous ophthalmic solution had a pH of about 4.5 to 5.5 and an osmolality of about 117 mOsm/kg.

Measurement of Osmolality—Transfer 2 ml of sample into 5 ml volumetric flask and add mili-Q water up to mark. Mix well and measure osmolality of sample using Osmometer (Advanced Instruments, Model 3250) with the working principle of freezing point depression; ensure that there are no air bubble during analysis.

The physicochemical stability of the aqueous ophthalmic solution obtained by the improved process was tested upon storage at room temperature (25° C./40% relative humidity), at 2-8° C. for 24 months and under accelerated storage conditions (40° C./25% relative humidity) for 6 months. Surprisingly, it was found that the aqueous solution remained clear and free from particles, crystals or precipitate upon storage. The assay of difluprednate remained in the range of 95%-105% w/v, the known and unknown impurities did not increase substantially upon storage and the content of impurities remained below the desired specified limit.

1. A process for the preparation of an aqueous ophthalmic solution of difluprednate having a pH of 4.5 to 5.5 and an osmolality from about 95 to about 150 mOsm/kg, comprising:
a. preparation of a non-aqueous drug phase by dissolving difluprednate and benzalkonium chloride in polyoxyl 35 castor oil;
b. preparation of an aqueous phase comprising:
i. preparing a polymer phase by dissolving polyvinyl alcohol in water for injection;
ii. preparing a buffer phase by dissolving glacial acetic acid, glycerin, boric acid, disodium edetate, sodium acetate and N-lauryl sarcosine sodium in water for injection at a stirring rate of about 200 to about 400 rotations per minute; and
iii. addition of the buffer phase to the polymer phase, wherein the addition is done at a stirring rate of about 200 to about 600 rotations per minute;
c. preparation of a bulk solution by addition of the non-aqueous drug phase to the aqueous phase; and
d. addition of polyhexamethylene biguanide to the bulk solution to achieve the aqueous ophthalmic solution of difluprednate.

2. The process according to claim 1, wherein the process further comprises filtration of the aqueous ophthalmic solution of difluprednate through a sterile 0.2 micron filter.

3. The process according to claim 1, wherein the addition of N-lauryl sarcosine sodium in the buffer phase preparation is done at a stirring rate of about 200 to about 400 rotations per minute.

4. The process according to claim 1, wherein the aqueous ophthalmic solution of difluprednate comprises:
i. about 0.03% w/v to about 0.04% w/v difluprednate;
ii. about 0.5% w/v to about 3.0% w/v polyvinyl alcohol or derivative thereof;
iii. about 0.01% w/v to about 0.05% w/v benzalkonium chloride; and
iv. about 1.5% w/v to about 6.0% w/v polyoxyl 35 castor oil.

5. An aqueous ophthalmic solution of difluprednate having a pH of 4.5 to 5.5 and an osmolality from about 95 to about 150 mOsm/kg, comprising:
i. about 0.03% w/v to about 0.04% w/v difluprednate;
ii. about 0.5% w/v to about 3.0% w/v polyvinyl alcohol or derivative thereof;
iii. about 0.01% w/v to about 0.05% w/v benzalkonium chloride; and
iv. about 1.5% w/v to about 6.0% w/v polyoxyl 35 castor oil;
wherein said aqueous ophthalmic solution is prepared by the process comprising:
a. preparation of a non-aqueous drug phase by dissolving difluprednate and benzalkonium chloride in polyoxyl 35 castor oil;
b. preparation of an aqueous phase comprising:
i. preparing a polymer phase by dissolving polyvinyl alcohol in water for injection;
ii. preparing a buffer phase by dissolving glacial acetic acid, glycerin, boric acid, disodium edetate, sodium acetate and N-lauryl sarcosine sodium in water for injection at a stirring rate of about 200 to about 400 rotations per minute; and
iii. addition of the buffer phase to the polymer phase, wherein the addition is done at a stirring rate of about 200 to about 600 rotations per minute;
c. preparation of a bulk solution by addition of the non-aqueous drug phase to the aqueous phase; and
d. addition of polyhexamethylene biguanide to the bulk solution to achieve the aqueous ophthalmic solution of difluprednate.

6. An aqueous ophthalmic solution of difluprednate having a pH of 4.5 to 5.5 and an osmolality from about 95 to about 150 mOsm/kg.

7. The aqueous ophthalmic solution of difluprednate according to claim 6 comprises:
i. about 0.03% w/v to about 0.04% w/v difluprednate;
ii. about 0.5% w/v to about 3.0% w/v polyvinyl alcohol or derivative thereof;
iii. about 0.01% w/v to about 0.05% w/v benzalkonium chloride; and
iv. about 1.5% w/v to about 6.0% w/v polyoxyl 35 castor oil.

8. The aqueous ophthalmic solution of difluprednate according to claim 7 further comprises:
v. about 0.002% w/v to about 0.02% w/v polyhexamethylene biguanide;
vi. about 0.02% w/v to about 0.05% w/v N-lauroyl sarcosine sodium;
vii. about 0.05% w/v to about 1.5% w/v boric acid;
viii. about 0.01% w/v to about 0.1% w/v disodium edetate; and
ix. about 1.0% w/v to about 3.0% w/v glycerine.

9. The aqueous ophthalmic solution of difluprednate according to claim 6, prepared by the process comprising:
a. preparation of a non-aqueous drug phase by dissolving difluprednate and benzalkonium chloride in polyoxyl 35 castor oil;
b. preparation of an aqueous phase comprising:
i. preparing a polymer phase by dissolving polyvinyl alcohol in water for injection;
ii. preparing a buffer phase by dissolving glacial acetic acid, glycerin, boric acid, disodium edetate, sodium acetate and N-lauryl sarcosine sodium in water for injection at a stirring rate of about 200 to about 400 rotations per minute; and
iii. addition of the buffer phase to the polymer phase, wherein the addition is done at a stirring rate of about 200 to about 600 rotations per minute;
c. preparation of a bulk solution by addition of the non-aqueous drug phase to the aqueous phase; and
d. addition of polyhexamethylene biguanide to the bulk solution to achieve the aqueous ophthalmic solution of difluprednate.
